(19) United States
(12) Patent Application Publication
(10) Pub. No.: US 2008/0221415 A1
(43) Pub. Date: Sep. 11, 2008

(54) Systems and methods for measuring and improving blood chemistry

(75) Inventor: David A. Sweeney, Hayward, CA (US)

Correspondence Address: KLARQUIST SPARKMAN, LLP, 121 SW SALMON STREET, SUITE 1600, PORTLAND, OR, 97204, US

(73) Assignee: Shaklee Corporation

(21) Appl. No.: 11/891,042

(22) Filed: Aug. 7, 2007

Related U.S. Application Data
(60) Provisional application No. 60/836,534, filed on Aug. 8, 2006.

(51) Int. Cl. A61B 5/00

(52) U.S. Cl. 600/316; 600/322

(57) ABSTRACT

Measurement systems provide a determination of relative concentrations of biological analytes based on transmission or reflection of near-infrared radiation by an in vivo specimen. Concentration and concentrations ratios associated with (ω-3, ω-6, and ω-9 fatty acids, lipids, glycosylated proteins, blood glucose, and cholesterol can be determined, and based on the determination an indication of subject health can be provided, or a dietary recommendation can be made. In one example, ratio of a concentration of ω-3 fatty acids to a combined concentration of ω-6 and ω-9 concentrations is determined. Dietary supplements can be recommended or ordered from a supplier based on the concentrations and concentration ratios.

### CROSS-REFERENCE TO RELATED APPLICATIONS

The present application claims the benefit of U.S. Provisional Application 60/836,534, filed Aug. 8, 2006, that is incorporated herein by reference.

### TECHNICAL FIELD

The disclosure pertains to analytical methods and systems for measuring or determining properties and/or amounts of analytes in living subjects.

### BACKGROUND

Laboratory analyses of biological specimens such as blood or urine samples are routinely used by medical professionals to diagnose and treat disease. These analyses generally require extraction of a specimen from a patient for subsequent processing and analysis. In some applications, specimen extraction can be painful, and sedation can be required. Some analyses have been incorporated into systems for individual use without assistance from medical personnel. Examples of such analyses include glucose testing for diabetics and pregnancy test kits. Nevertheless, most analytical procedures remain useful only in laboratory settings. As a result of the cost, complexity, and discomfort associated with laboratory analyses, today's sophisticated laboratory analyses are not useful for personal health self-assessments. As a result, many potential indicators of personal health remain unused. Accordingly, improved methods and apparatus are needed.

### SUMMARY

Representative methods of providing a health assessment comprise emitting radiation towards a living biological sample of a subject and collecting data corresponding to an interaction of the emitted radiation with the living biological sample. Concentrations and/or a ratio of a concentration of at least a first biological analyte to a concentration of a second biological analyte based on the collected data is determined. Concentrations and associated ratios can be determined for a plurality of analytes. An indication of subject health is provided based on the ratio, ratios, or concentrations. In some examples, the emitted radiation is near-infrared radiation and the collected data is based on a portion of the emitted radiation transmitted or reflected by the sample. In other examples, the emitted radiation is near-infrared radiation in a wavelength range between about 650 nm and 2700 nm and the determined ratio is associated with ω-3 fatty acids or a combined concentration of ω-6 and ω-9 fatty acids. In further representative examples, the determined ratio is a ratio of a concentration of ω-3 fatty acids to a concentration of ω-6 fatty acids. In other examples, levels of lipids, glycosylated proteins, blood glucose, and cholesterol are determined. In some examples, the health assessment is associated with a human subject, but health assessments can also be provided for veterinary applications.

Health assessment apparatus comprise a radiation source configured to emit electromagnetic radiation and direct the emitted radiation to an in vivo sample. A detector is configured to detect radiation associated with an interaction of the emitted radiation with the in vivo sample and provide an associated detection signal. A processor is configured to receive the detection signal and determine a ratio of concentrations of a first biological analyte to a second biological analyte in the in vivo sample based on the detection signal. An indication associated with the ratio is provided by a display. In some examples, the processor is configured to store sample data associated with the detection signal in a memory. In other examples, the memory is configured to store reference data associated with at least one of the first and second biological analytes, and the processor is configured to produce the indication associated with the ratio based on the stored sample data and the stored reference data. In additional examples, the first biological analyte includes ω-3 fatty acids and the second biological analyte includes ω-6 fatty acids and ω-9 fatty acids. In other examples, concentrations, ratios of concentrations, or other levels associated with lipids, glycosylated proteins, blood glucose, and cholesterol can be determined. In some examples, the radiation source is configured to emit near infrared radiation, and the in vivo sample is situated to reflect or transmit the detected radiation to the detector. In other examples, a sample holder is configured to position the in vivo sample with respect to the radiation source and the detector. In further representative embodiments, the processor is configured to determine the ratio based on emitted radiation in a wavelength range between about 1150 nm and 1190 nm. In other examples, a wand is configured to provide radiation and direct radiation from the sample to the detector. In some examples, the wand can include one or both of the radiation source and the detector, and can be configured to direct transmitted or reflected radiation to the detector.

According to some aspects of the disclosed technology, methods comprise non-invasively scanning a living organism to determine an amount of ω-3 fatty acids in the living organism with respect to a reference analyte. An indication of an amount of ω-3 fatty acids available in an ingestible substance is obtained, and a dietary recommendation for the living organism is provided relating to ingestment of the ingestible substance by the living organism based on the determined amount of ω-3 fatty acids in the living organism and in the ingestible substance. In further examples, an amount of the reference analyte in the ingestible substance is determined, wherein the dietary recommendation is based on the amount of the reference substance in the living organism and the ingestible substance. In some examples, the reference substance consists essentially of at least one or both of ω-6 fatty acids and ω-9 fatty acids. In other examples, amounts of lipids, glycosylated proteins, blood glucose, and/or cholesterol are assessed with respect to reference levels, and corresponding dietary recommendations and supplements can be provided.

Methods are disclosed that include providing an analyte measurement apparatus to a dietary supplement customer, and producing a supplement recommendation based on in vivo assessment of a plurality of analytes produced by the apparatus. Supplements are provided based on the supplement recommendation. In some examples, at least one of the plurality of analytes is selected from the group consisting of lipids, glycosylated proteins, blood glucose, and cholesterol. In other examples, the plurality of analytes include lipids, glycosylated proteins, and cholesterol. In additional representative examples, a health indicator is provided based on the in vivo assessment. In additional examples, the in vivo assessment is based on at least one near infrared spectrum.

These and other features and aspects of the disclosed technology are set forth below with reference to the accompanying drawings.

### BRIEF DESCRIPTION OF THE DRAWINGS

FIG. 1 is a schematic diagram of a representative system for measuring a biological analyte or a ratio of such analytes.

FIG. 2 is a schematic diagram of another representative system for measuring a biological analyte or a ratio of such analytes.

FIG. 3 is a schematic diagram of a representative system for determining a chemometric or calibration method for determining analyte concentrations for a previously uncalibrated method based on a comparison of measurement results obtained with a conventional analytical method for a particular analyte.

FIG. 4 is graph of absorbance as a function of wavelength for several types of oils that include peaks that can be associated with ω-3, ω-6, and ω-9 fatty acids.

FIG. 5 is a portion of the graph of FIG. 4 illustrating spectral features associated with ω-3 contributions.

FIG. 6 is a schematic diagram of an apparatus for in vivo measurement of biological analytes or ratios of concentrations of biological analytes.

FIG. 7 is a schematic diagram of a measurement apparatus configured to provide estimates of concentrations of a plurality of disparate analytes in an in vivo specimen and communicate measurement results to a user or to a supplement provider via a network.

### DETAILED DESCRIPTION

As used in this application and in the claims, the singular forms “a,” “an,” and “the” include the plural forms unless the context clearly dictates otherwise. Additionally, the term “includes” means “comprises.” The described systems, apparatus, and methods described herein should not be construed as limiting in any way. Instead, the present disclosure is directed toward all novel and non-obvious features and aspects of the various disclosed embodiments, alone and in various combinations and sub-combinations with one another. The disclosed systems, methods, and apparatus are not limited to any specific aspect or feature or combinations thereof, nor do the disclosed systems, methods, and apparatus require that any one or more specific advantages be present or problems be solved.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed systems, methods, and apparatus can be used in conjunction with other systems, methods, and apparatus. Additionally, the description sometimes uses terms like “produce” and “provide” to describe the disclosed methods. These terms are high-level abstractions of the actual operations that are performed. The actual operations that correspond to these terms will vary depending on the particular implementation and are readily discernible by one of ordinary skill in the art.

Analyte measurements are described below with reference to analyte concentrations. In some examples, concentrations, total analyte amounts, or other level indicators can be used, and concentrations are merely one example. In addition, while in vivo measurements are particularly convenient, laboratory or in vitro measurements can also be used.

Information about biological analytes can be obtained using many diverse methods, typically based on methods associated with analytical chemistry. Some of these techniques are invasive, and some are non-invasive to a living organism. Practical uses with human subjects can depend on the invasiveness of a particular method. Samples for analysis can be difficult to obtain, especially if obtaining a sample involves an invasive technique that requires, for example, puncturing the skin of a patient. In some embodiments, a sample is removed from a subject and analyzed ex-vivo while in some advantageous embodiments, a sample is not removed from a subject, and the sample can be analyzed in-vivo. Such in-vivo-based analyses are typically preferred by subjects, especially if the analysis is sufficiently straightforward that a clinician is unnecessary for the analysis.

In-vivo measurement of concentrations or ratios of concentrations of complex molecules such as fatty acids is generally necessary to avoid subject pain or other discomfort. Furthermore, it can be difficult even in a laboratory setting to correlate complex ratios of analytes to the health of an organism. Systems and methods disclosed herein can be used in solving such problems.

Many techniques involve actively emitting radiation toward a solution or other specimen and detecting transmitted or reflected radiation or both as a function of radiation wavelength. Such measurements can provide information about substances in the sample, including analytes of interest. Typical techniques involve detecting radiation that is directed to the sample with a dedicated source and that is either transmitted or reflected by the sample, or by detecting ambient radiation transmitted or reflected (or both (by the sample. In some examples, sample information is obtained for a large number of wavelengths (typically several or many hundreds of wavelengths), and chemometric or other calibration or analysis methods are used to correlate this information with concentrations of one or more analytes. Some representative methods are based on multi-wavelength, multi-analyte analyses that can be conveniently executed for in vivo samples using near infrared radiation.

Such spectroscopic techniques can use various kinds of electromagnetic radiation, and in some techniques radiation that includes a broad band of wavelengths is directed toward a sample. These techniques can use near-infrared (NIR), mid-infrared (MIR), and/or far-infrared (FIR) radiation, for example. Narrow spectrum techniques can be based on radiation in a narrower band of wavelengths such as typically provided with a laser. Mathematical tools can also be used to process detected energy, as with Fourier-transform infrared (FTIR) techniques. Near-infrared based techniques can be particularly convenient in evaluations of fatty acids. Representative NIR techniques for in vitro samples are described in, for example, FT-IR Technical Note TN002, NIR Technologies, Inc., and Sato, Biosci. Biotechnol. Biochem. 66:2543-2548 (2002) that are both incorporated herein by reference.

Detection of analytes of interest such as ω-3 fatty acids can be difficult in practical settings and identification of a particular reference specimen or reference model for comparison with a measured spectrum can enhance measurement speed, accuracy, and reliability. Spectra of many fatty acids exhibit similar, broad spectral features and methods other than direct spectral comparison can be helpful. For example, second derivative spectra tend to facilitate identification and quantification of different fatty acids due to the relatively larger differences in second derivative spectra than ordinary absorbance spectra. In some examples, second derivative spectra can be obtained based on moving average spectral values, a size of derivative segments, and a gap between derivative segments. Examples of such processing are described in, for example, the Sato article referenced above. Typically, second derivative spectra are based on second derivatives of absorbance spectra. Alternatively, factor analysis such as principal component analysis can be used to distinguish analytes or groups of analytes in addition to or instead of direct comparisons of absorbance spectra.

In some examples, measurements are directed to assessing health of a living organism such as a human or other animal. Health assessment can be associated with evaluation of nutrient concentrations or concentrations of other substances including subject tissues. For example, body fat, muscle mass, cholesterol levels, ratios thereof, or other features can be quantified.

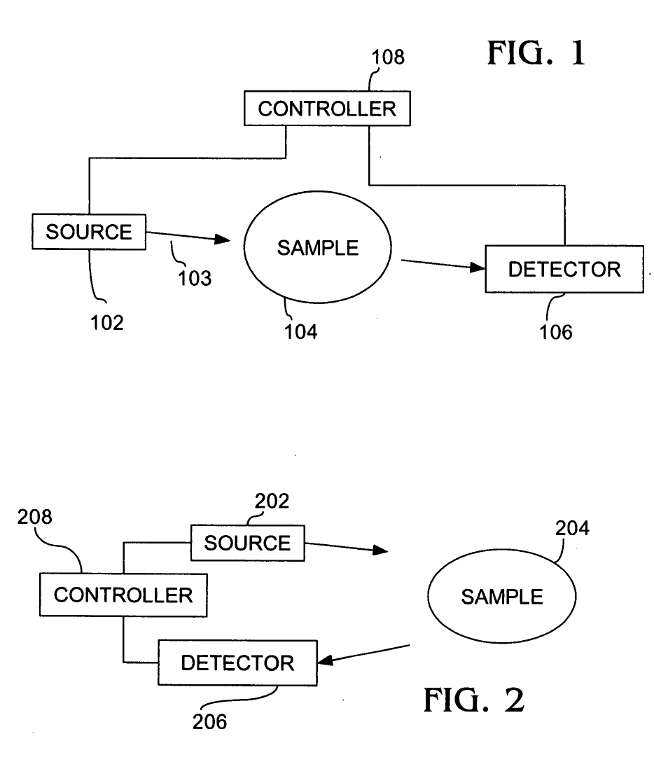

FIG. 1 is a schematic diagram of a representative transmission-based system that includes a radiation source 102 configured to direct a radiation flux 103 to a sample 104. A detector 106 is situated to receive radiation transmitted by the sample 104. The source 102 can be selected so as to emit electromagnetic radiation at any wavelength or in any wavelength range such as, for example, visible, ultraviolet, infrared, or other ranges. In some embodiments, the source is a laser, one or more light emitting diodes, or other coherent or incoherent light source. In some advantageous embodiments, the source emits infrared radiation generally in the near-infrared range. A controller 108 can be coupled to the radiation source 102 and the detector 106 and configured to store acquired data such as absorbance spectra in a memory or computer-readable medium such as read only memory (ROM), random access memory (RAM), a hard disk, a floppy disk, or other type of memory or data storage device.

FIG. 2 is a schematic diagram of a representative reflection-based system for sample investigation. A radiation source 202 is configured to direct a radiation flux to a sample 204 and a detector 206 is situated to receive radiation reflected by the sample 204. Various sources and/or detectors can be used. In some embodiments, the source 202 emits radiation in a broad range of wavelengths and the detector 204 is configured to detect radiation at a single wavelength or in a narrow range of wavelengths. In some embodiments, the source 202 emits radiation in a narrow range of wavelengths and the detector detects radiation in a broader range of wavelengths. In some embodiments, the source 202 and detector 206 are tuned to emit and detect radiation having wavelengths in generally the same range. A controller 208 is coupled to the radiation source 202 and the detector 206 and can be configured to store and process data such as spectral data. Other representative systems include the configurations of FIG. 1 and FIG. 2 and can detect transmitted radiation, reflected radiation, or both. In some examples, multiple sources and/or multiple detectors are used, and spectrally selective components such as diffraction gratings, prisms, or holograms are situated to permit measurement of absorbance or other specimen property as a function of radiation wavelength or to reject unwanted radiation.

As shown in FIGS. 1-2, the controllers 108, 208 are configured to control, for example, one or both of the source and/or detector. In some embodiments, a controller can control the type or amount of radiation emitted by the source and/or the type or amount of radiation detected by the detector. In some embodiments, a controller can be used to store or control the storage of data received by the detector. A controller can comprise or be in communication with a computer that can include a database for storing such data and/or a processor for processing such data. The controller can also be coupled so as to communicate with a computer such as a desktop, laptop, or palm top computer for storage and analysis of measurement results via a network or other wired or wireless connection. Alternatively, the controller can be configured to direct data or other information to a removable memory medium for transport to another computer or other device. For example, in some applications, a user of systems such as those of FIGS. 1-2 can record measurement results as a function of time, diet, exercise, or other parameters, and the user can transfer such results to a personal computer or personal digital assistant or other device via network, or using a removable storage medium.

Samples such as those investigated with the systems of FIGS. 1-2 can be biological tissue that is living or formerly living, for example. A sample can be a bodily fluid or a component thereof such as whole blood, blood serum, interstitial fluid, saliva, sweat, urine, mucus, spinal fluid, and/or lymphatic fluid, for example, or any combination thereof. The sample can be a living human finger or a portion thereof. The sample can be any portion of a human's skin or other tissue. In some embodiments, the sample can comprise an ear-lobe, a gill, a fingernail, webbing between appendages such as fingers or toes, a fin, a tail, an ear, eyelid, skin flap, umbilical cord, tongue, etc. In some embodiments, preferred samples can be any thin tissue that is highly vascularized, for example, or near to bone. In other examples, other human or animal tissues or parts, can be evaluated. In addition, nutrients in human or animal tissues, or in foodstuffs such as fruits, meats, vegetables, or other foods or food supplements can be evaluated.

Typical samples of interest can contain one or multiple analytes such as fatty acids, ω-3 fatty acids, ω-6 fatty acids, ω-9 fatty acids, antioxidants, vitamin E, glycolysated proteins, lipofuscin, glucose, hemoglobin, hematocrit, sugars, proteins, cellular matter, nutrients, free radicals, chemicals, lipids, phospholipids, lipoproteins, chromosomes, telomeres, mitochondria, sub-cellular organelles, vesicles, RNA, DNA, protein complexes, nutritional products, vitamins, minerals such as magnesium and in bone, polyphenols including but not limited to flavinoids, catechins, proanthocyanidins, anthocyanidins and derivatives thereof, etc. In some embodiments, the analyte can be a combination of any of these substances.

In other examples, analytes of interest are indicative of nutritional deficiency, nutritional depletion, nutritional adequacy, nutritional repletion, or other nutritional condition of a human or animal. Other analytes are indicative of impaired biological structure, enhanced biological structure, or other structural conditions. Analytes can also include markers of biological function that are indicative of enhanced (or depressed) biological function such as, for example, an ergogenic aid such as creatine.

While some examples are associated with human or animal health, the disclosed methods and apparatus can also provide indications of ω-3, ω-6, ω-9 fatty acids or other nutritional analytes in foodstuffs such as eggs, butter and meat. Livestock can be scanned to determine, for example, acceptable ω-3 content prior to slaughter, determining whether animals are ready for slaughter, or as an aid in selecting feed or other treatment or care. In addition, while examples are described for use with living humans, the disclosed methods and apparatus are also suitable for in vitro use with samples that are not living. Body composition (body fat, muscle mass) of humans or animals can also be evaluated.

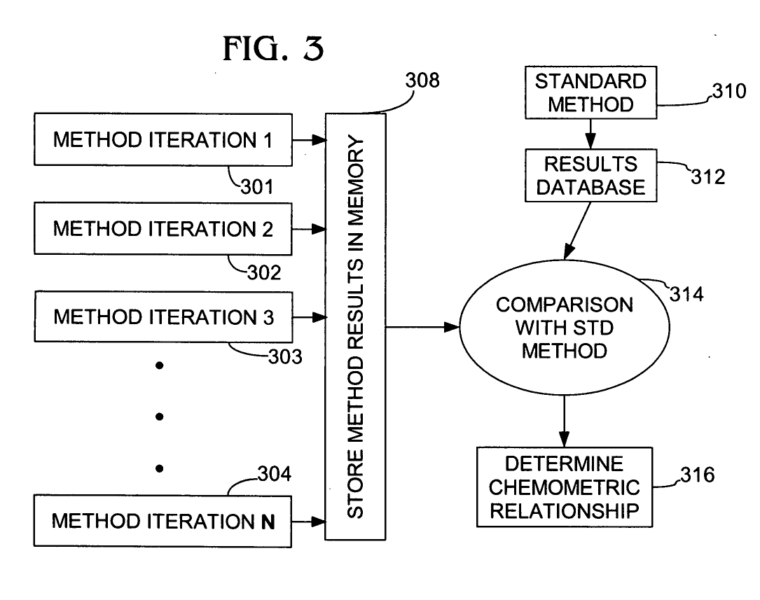

FIG. 3 is a block diagram that illustrates representative systems and methods for determining a relationship between measurement results or predictions or other data from a new or previously uncalibrated or untested measurement method and a result of a conventional method or other typically used method. Such an analytical approach can be referred to as a “chemometric” analysis. Chemometrics can comprise any application of mathematical or statistical methods to chemical data such as, for example, use of neural networks or equivalents thereof to derive or discover differences between data sets over a broad or narrow spectrum. Method iterations 301, 302, 303, 304 can be based on scanning a sample with NIR energy in first, second, third, and Nth wavelength ranges, respectively, (or at respective wavelengths) and detecting corresponding (e.g., reflected or transmitted) energies or powers, typically as a function of wavelength or wavelength range. This process can be repeated for many wavelengths. In some embodiments, this process is repeated for 300 or more wavelengths or wavelength ranges but few or many hundreds of wavelengths can be used in some examples. As shown in FIG. 3, results of such processes are stored in a database in step 308 that can be provided in a memory such as RAM, ROM, a disk drive, or other computer-writable medium.

A standard method 310 can also be employed independently of the various iterations of the new method. The standard method 310 can be used to find a result that can be used for comparison and/or calibration of the new method. In some embodiments, the known method can be a bioanalytical chemistry method such as high performance liquid chromatography (HPLC), invasive FTIR, chemical electrophoresis (CE), mass spectrometry, or other methods. Such standard results are stored in a step 312.

The result of the known method can be compared sequentially or in parallel with results of the new method or various combinations thereof in a step 314. For example, a computational computer program can be used to find the best fit to a curve or a surface represented by a matrix of coefficients representing the values and/or data obtained through the various iterations of the new method. This computation can provide a formula, a relationship, and/or a combination of the results that provide statistically significant correlations between the results of the new method and the result of the known method. Based on this computation, a chemometric relationship is established in a step 316.

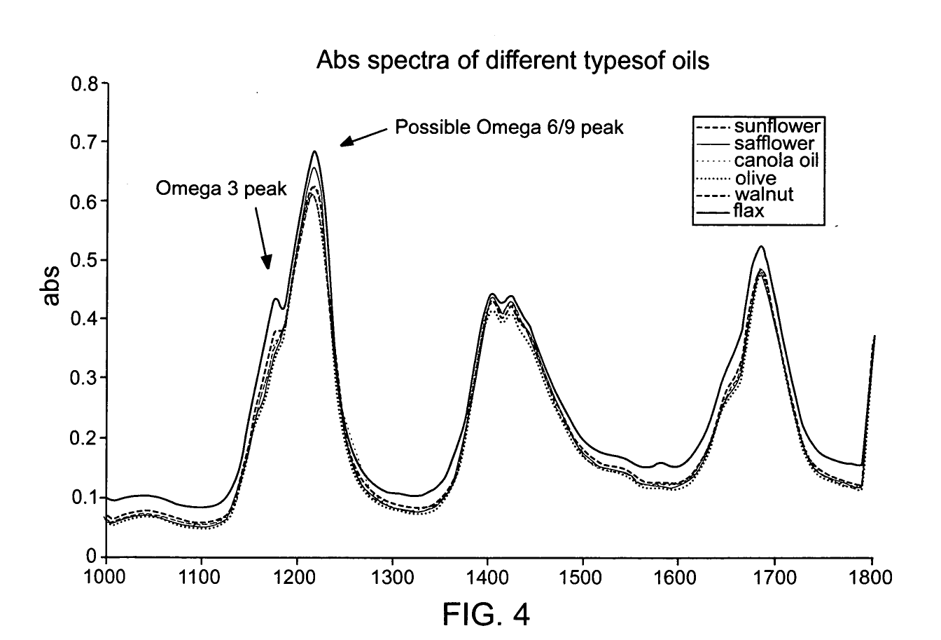

FIG. 4 illustrates spectroscopic absorbance data for various analytes that have been scanned using near-infrared (NIR) spectroscopic techniques. An ω-3 peak (data maximum indicating presence and/or amount of ω-3 fatty acid in the analyte) is labeled, as well as a possible ω-6/ω-9 peak. Other peaks or spectral features can also be used in conjunction with chemometric models for prediction of concentrations and concentration ratios of various analytes of specimens of interest. The absorbance spectra plotted in FIG. 4 correspond to different types of oils, including sunflower oil, safflower oil, canola oil, olive oil, walnut oil, flax oil. These substances are important to measure because fatty acids in oils play a significant role as a food source for humans. Excessive dietary fat intake has been linked to obesity, coronary heart disease and high cholesterol levels in the blood serum. In addition, a particular group of fatty acids has been shown to have many industrial uses in, for example, ink and paint manufacturing.

Ratios of concentrations or quantities of various analytes can be used to assess subject health, diet, or to evaluate foods or dietary supplements. For example, a ratio of ω-6 fatty acids to ω-3 fatty acids in food can be an indicator of how healthy the food is; similarly, the ratio of ω-6 fatty acids to ω-3 fatty acids in a person's blood stream and adipose tissue can serve as an indicator of the health of the person. Thus, it can be highly advantageous to measure the concentrations, independent amounts, and/or ratios of concentrations or amounts of these two substances in the body (e.g., in the blood stream or other bodily fluid). Furthermore, it can be highly advantageous to determine whether or not a person's diet and/or dietary supplements have succeeded in producing a desired amount, concentration, and/or ratio of a certain substance or substances (e.g., ω-6 fatty acids and ω-3 fatty acids).

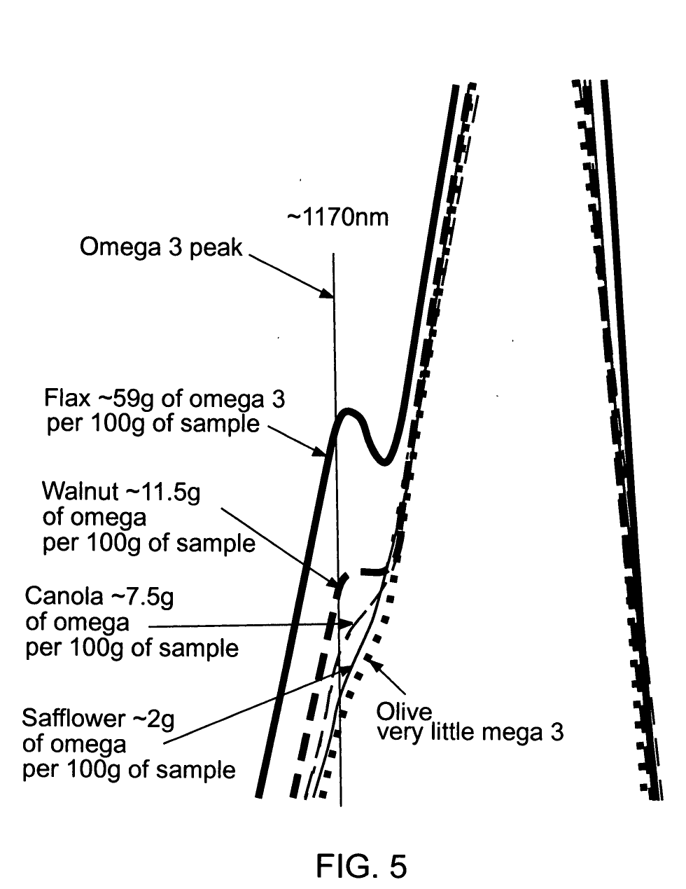

FIG. 5 illustrates a portion of the spectroscopic absorbance data of FIG. 4. As is apparent from FIG. 5, differences in ω-3 fatty acid concentrations can be observed at wavelengths around 1170 nm. For some oils, there are readily discernable ω-3 peaks. Peak size can be directly and/or approximately correlated to ratios of ω-3/ω-6, and 50-fold changes in ratios can be observed. In some examples, one, two or many spectral features or spectral portions can be used, and the spectroscopic data of FIGS. 4-5 represents only a simple illustrative example.

Relative and/or absolute amounts of ω-3 fatty acids in a living organism can have a beneficial health effect, or can be an indicator of good health. For example, increasing intake of ω-3 fatty acids relative to ω-6 fatty acids and/or ω-9 fatty acids can be beneficial. Ingestible substances such as food or dietary supplements, including, for example, those containing fish oil or flax seed oil, can contain relatively large amounts of ω-3 fatty acids and can provide increased ω-3 fatty acid intake. Most human diets are less rich in ω-3 fatty acids, however, and typically include more oils with abundant ω-6 fatty acids. Olive oil has higher amounts of ω-9 fatty acids. Some studies show that the usual ratio of ω-6 fatty acids to ω-3 fatty acids is approximately 10/1. However, it is beneficial to have ratios of approximately 4/1 or less. This ratio can be decreased by decreasing the numerator (amount of ω-6 fatty acids) or by increasing the denominator (amount of ω-3 fatty acids). One way of changing this ratio is by ingesting or otherwise introducing ω-3 fatty acids, or the precursors thereof, into a living organism. Humans can accomplish this, for example, by ingesting more fish oils or appropriately designed nutritional supplements, for example. In some embodiments, various ratios can be measured and controlled, and appropriate dietary changes made or nutritional supplements provided. For example, the following fatty acid ratios can be of interest: ω-6/ω-3; ω-3/ω-6; (ω-6+ω-9)/ω-3; ω-6/ω-3+ω-9); ω-9/ω-3; ω-3/ω-9; ω-6/ω-9; ω-9/ω-6 but many other ratios and combinations are also possible. Indeed, more complex relationships such as an (ω-6/ω-3)/vitamin C ratio are also potential indicators of good health. Other concentration or composition ratios that can be analyzed and/or used to improve health or to provide indications of health include: 1) ratios of saturated fatty acids to mono- and/or polyunsaturated fatty acids and permutations thereof; 2) specific ratios of different fatty acids such as oleic acid to palmitic acid and others described in Pacheco et al., Am. J. Clin. Nutr. 84:342-349 (2006) and Vega-Lopez et al., Am. J. Clin. Nutr. 84:54-62 (2006) both of which are incorporated herein by reference, a ratio of n-6 to n-3 fatty acids and ratios of “good” cyclooxyenase precursors (eicosapentoic acid (EPA) and dihomogammalinoleic acid (DGLA)) to “bad” ones (like AA) as described in Miljanovic, Am. J. Clin. Nutr. 82-887-893 (2005) that is incorporated herein by reference, 3) ratios of ω-3 fatty acids (or a ω-3 vs. ω-6 ratio) to HDL, LDL, TGs, subclasses of HDL, etc. (HDL subclasses are described in the Vega-Lopez article cited above); 4) ratios of ω-3 fatty acids to inflammatory markers such as various classes of prostaglandins, tumor necrosis factor (TNF), nitric oxide, nuclear factor Nfkappabeta, etc.

The biophysical explanation underlying the health benefits of lower ω-6/ω-3 fatty acid ratios may relate to the double bonds that hold portions of the lipid molecules together. Some health benefits may relate to displacement of arachidonic acid as the major substrate for cyclooxygenase (and therefore a better profile of anti-inflammatory/inflammatory eicosinoids). Some health benefits may relate to modulation of transcription factors for pro- and anti-inflammatory pathways, and direct enzyme inhibition, etc. Some of these mechanisms are discussed in Simopoulis, J. Am. C. Nutr. 21:495-505 (2002) and the previously cited Miljanovic reference. Some health benefits are also discussed in Wang et al., Am. J. Clin. Nutr. 84:5-17 (2006) and more information concerning fatty acids can be found on the Internet at en.wikipedia.org/wiki/Essential_fatty_acid_interactions, both of which are incorporated herein by reference.

Spectra that can be processed to determine analyte concentrations, concentration ratios or other functions of analyte concentration can be obtained using, for example, measurements of transmitted or reflected optical power in a predetermined spectral range that is selected with, for example, one or more prisms, diffraction gratings, or holographic optical elements that disperse the transmitted or reflected optical power to one or more detector elements as a function of wavelength. Alternatively, so-called Fourier transform spectroscopy can be used. In typical examples, optical radiation in a so-called near infrared (NIR) wavelength range that extends from about 650 nm to about 1800 nm can be used. NIR wavelengths can be especially convenient for in vivo human applications for because radiation at such wavelengths can be effectively transmitted through body parts. In one example of an in vivo method and apparatus, a NIR source directs a NIR optical beam to a body part such as a finger and transmitted light is captured and spectrally dispersed for delivery to one or more detectors. Typically, a detector array is provided, and radiation at selected wavelengths or in selected wavelength ranges is directed to particular detectors of the array of detectors. A transmitted spectrum based on the radiation received by the detector array can be stored in a memory and compared with a measured or estimated spectrum associated with the optical beam without interaction with the in vivo specimen.

In some examples, a measured spectrum is compared with a reference spectrum obtained by directing the NIR optical beam to a reference or calibration standard. Reference or calibration standards can be particularly useful in applications in which small changes in absorbance are used. Some representative temperature stabilized standards based on glass and PTFE are described in, for example, Samsoondar and Kaushal, U.S. Pat. No. 6,917,422 that is incorporated herein by reference. Dual beam measurement systems can be provided in which a specimen and a standard can be interrogated with different radiation beams.

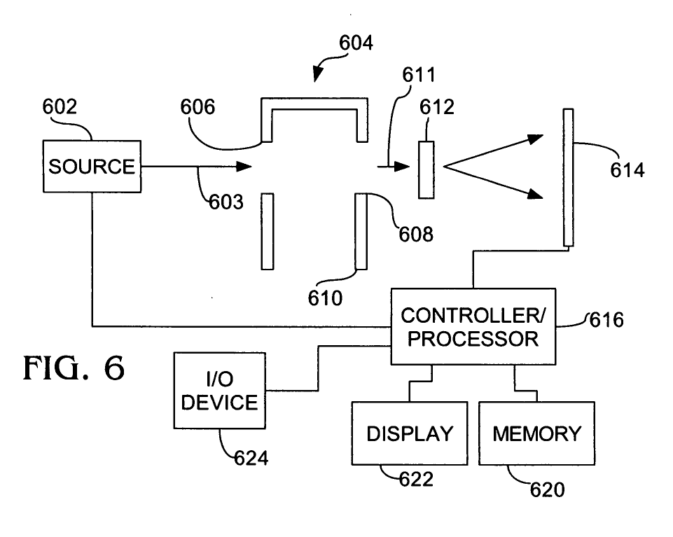

A representative optical interrogation system for determining ω-3/ω-6, ω-3/ω-9, and other concentration ratios is illustrated in FIG. 6. A radiation source 602 is configured to deliver a radiation flux 603 substantially at near-infrared wavelengths to a sample retainer 604 that is configured to receive a finger or other body part of a subject to be tested. The retainer includes a radiation entrance aperture 606, and exit aperture 608, and a finger insertion aperture 610. The sample retainer permits radiation from the source 602 to reach a sample, but shields the sample from ambient radiation. A diffraction grating 612 or other dispersive optical element receives a transmitted radiation flux 611 and directs the dispersed flux to a detector array 614.

The source 602 and the detector array 614 are coupled to a controller 616 that is in communication with a memory 620, a display 622, and a user input/output device 624. The controller 616 is configured to receive electrical signals from the detector array 614 and store in the memory 620 a representation of a transmission optical spectrum associated with the specimen and/or determine an absorbance spectrum. Typically, the controller 614 is further configured to estimate concentrations or concentration ratios of one or more fatty acids such as an ω-3/ω-6 ratio based on the recorded spectrum. The display 622 can be configured to provide a numerical readout associated with concentrations or concentration ratios, or a bar graph of other indication associated with “good,” “bad,” and “intermediate” values.

In some example, chemometric, calibration, or other processing methods applied to collected data such as absorbance spectra are based on consideration of a distribution of one or more analytes in a subject. For example, an analyte can have different distributions in different subject compartments, and processing methods can be configured to provide compartment specific results or compartmental averages. Such compartmental results can also include proportions of a specimen that correspond to various compartments. For example, a particular analyte can have different distributions in blood, in interstitial fluid, and within cells. Concentrations in each of these compartments can be different, and calibration or other processing algorithms can be configured to provide compartment specific values or compartment averages.

As shown in FIG. 6, the sample retainer 604 is generally configured for the insertion of a body part (such as a finger) of a subject. In other examples, sample retainers can be configured to clamp or otherwise press against a body part. Such clamping or pressing can alter a compartmental composition of the measured specimen. Such alterations can be used to preferentially select or avoid a particular compartment. For example, pressure can reduce blood volume in the specimen, and hence reduce or eliminate a blood-related measurement contribution. In addition, volume changes in a specimen resulting from, for example, blood flow in in vivo specimens can be detected or compensated so as to provide consistent measurement results with or without consideration of changing compartmental distribution.

In some examples, skin specimens are evaluated. Representative devices for measuring compounds within skin and clamping devices for contacting skin specimens are described in U.S. Patent App. Pub. 2005/0075546 which is incorporated herein by reference. Non-invasive measurement systems for blood constituents are described in U.S. Pat. No. 5,361,758, U.S. Pat. No. 6,236,047, U.S. Pat. No. 6,040,569, and MacIntyre et al., U.S. Patent Application Publication 2007/0110621 that are incorporated herein by reference.

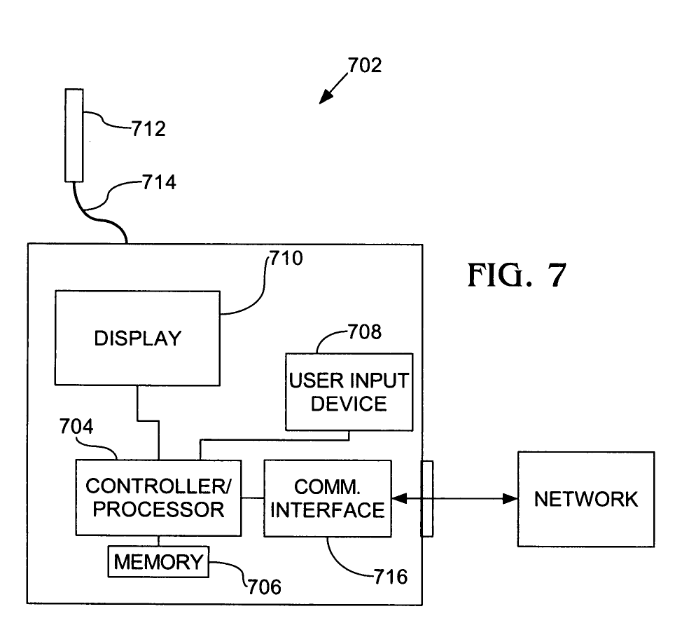

While representative non-invasive near-infrared based measurements are described in the above examples for selected analytes such as ω-3, ω-6, and ω-9 fatty acids, disparate analytes and associated ratios can be determined as well. For example, concentrations or other levels of lipids, glycosylated proteins, cholesterol, triglycerides, hemoglobin A1c (Hb A1c), or blood glucose can be measured. Ratios associated with concentrations or levels of, for example, ω-3/ω-6ω/Hb A1c can be determined. Determinations can be made for a plurality of analytes, and based on the determinations, one or more health indicators can be provided. In some examples, a health indication can be provided as a single number or other single dimensional representation such as a letter grade, or as a multidimensional representation such as an array. Analysis is not restricted to any particular tissues, and analytes found in bone, skin, blood, and other tissues or at different locations can be evaluated With reference to FIG. 7, a representative health assessment apparatus 702 includes a processor 704 that is coupled to a memory 706, a user input device 708 such as a mouse or keyboard (or several such devices), and a communication interface 716 A measurement probe 712 is connected via an electrical or optical cable 714. The probe 712 can be configured to insertion into a body part to determine analyte levels. Alternatively, the apparatus 702 can be configured to measure body parts or portions that are inserted into a measurement receptacle as well.

Portable or fixed communication hardware or storage can be provided. For example, an Ethernet, 803.11g, Bluetooth, or other wired or wireless communication hardware can be provided. Alternatively, an interface such as a universal serial bus (USB) or other connection can be provided so that instruction and data can be provided to or received from a local or wide area network such as the Internet, or removable storage media such as flash or other memory or disks. As shown in FIG. 7, the measurement apparatus 702 is in communication with a network such as the Internet.

The measurement apparatus 702 can be configured to receive instructions or data that pertain to, for example, a selection of analytes and/or analyte ratios for measurement, a type of health index to be determined or a selection of a number of health indicators associated with a multidimensional health index, or a procedure for determining a health index. In addition, partial compositional data associated with foods and nutritional supplements can be provided from, for example, a supplier web page or otherwise provided. User specific data or procedures can also be received from, for example, user-specific entries at a supplier web site. Such data can be based on, for example, preferred user health indicators or user goals for such indicators, user height, weight, age, sex, or supplements likely to be currently available to the user at home or work. Instructions, data, and other operational methods and parameters can be supplied via the communication interface 716 or the user input device 708.

Measurement results for one or more analytes are typically processed to determine at least one health indicator. Based on the determined health indicator, recommended nutritional or dietary products can be provided to the user. In some examples, the system is configured to produce recommendations based on data associated with nutritional supplements or food products stored in the memory 702. For example, nutritional or other values of foods and/or supplements can be stored in the memory and updated as needed. In some examples, concentrations or quantities of particular food components are provided such as, for example, polyphenols. Alternatively, the system can initiate a connection to a network such as the Internet, and obtain recommendations from a supplier web site or a supplier representative based on analyte measurements. In some examples, a recommendation produced either locally at a measurement device or at a network location serves as a basis for a supplement order transmitted to a supplier. In response to this recommendation, one or more foods or supplements can be delivered or ordered.

Measurement apparatus such as the measurement apparatus 702 can be provided by a supplement vendor to customers to permit customers to perform health self-assessments. Based on the health assessments, customers can be provided with supplement recommendations. In some examples, such recommendations can be communicated to the supplement vendor via the measurement apparatus or entered at a vendor web page and serve as the basis for ordering the supplements. In this manner, supplement vendors can conveniently provide customized recommendations to product users and deliver appropriate products based on the recommendations.

The preceding examples are not to be taken as limiting the scope of the disclosed technology but are provided for convenient illustration. For example, chemometric analyses can be used to determine analyte ratios, or simple comparisons of absorbance spectra can be used. Applications to some particular analytes (ω-3, ω-6, and ω-9 fatty acids) are described in detail, but the disclosed technology can be applied to other analytes as well. Typically a plurality of analytes is investigated and one or more health indices and/or dietary or dietary supplement recommendations are generated. In view of the preceding, I claim all that is encompassed by the appended claims.

What is claimed is:

1. A method, comprising:
emitting radiation towards a living biological sample of a subject;
collecting data corresponding to an interaction of the emitted radiation with the living biological sample;
determining a concentration of at least a first biological analyte based on the collected data; and
providing an indication of subject health based on the concentration.

2. The method of claim 1, further comprising determining a concentration of at least a second biological analyte based on the collected data, wherein the indication of subject health is based on the concentrations of the first biological analyte and the second biological analyte.

3. The method of claim 1, wherein the indication of subject health is associated with at least one nutrient concentration.

4. The method of claim 2, wherein the emitted radiation is near-infrared radiation and one of the first and second analytes is ω-3 fatty acids.

5. The method of claim 4, further comprising determining a ratio of the concentrations of the first analyte and the second analyte, wherein the indication of subject health is based on the ratio.

6. The method of claim 5, wherein the ratio is a ratio of a concentration of ω-3 fatty acids to a combined concentration of ω-6 and ω-9 fatty acids or a total combined concentration of fatty acids.

7. The method of claim 5, wherein the determined ratio is a ratio of a concentration of ω-3 fatty acids to a concentration of ω-6 fatty acids or a ratio of a concentration of ω-6 fatty acids to a concentration of ω-3 fatty acids.

8. The method of claim 2, wherein at least one of the first and second biological analytes is selected from the group consisting of fatty acids, ω-3 fatty acids, ω-6 fatty acids, ω-9 fatty acids, antioxidants, vitamin E, glycolysated proteins, lipofuscin, glucose, hemoglobin, hematocrit, sugars, proteins lipids, glycosylated proteins, cholesterol, blood glucose, and triglycerides.

9. The method of claim 2, wherein at least one of the first and second biological analytes is hemoglobin A1c (Hb A1c).

10. An apparatus, comprising:
a radiation source configured to emit electromagnetic radiation and direct the emitted radiation to an in vivo sample;
a detector configured to detect radiation associated with an interaction of the emitted radiation with the in vivo sample and provide an associated detection signal;
a processor configured to receive the detection signal and determine a concentration of a first biological analyte associated with the in vivo sample based on the detection signal; and
a display configured to provide a health assessment indication associated with the concentration.

11. The apparatus of claim 10, wherein the processor is configured to determine a concentration of a second biological analyte based on the detection signal, wherein the health assessment indication is associated with the concentrations of the first and second biological analytes.

12. The apparatus of claim 11, further comprising a memory is configured to store reference data associated with at least one of the first and second biological analytes and sample data associated with the detection signal, and the processor is configured to produce the indication based on the stored sample data and the stored reference data.

13. The apparatus of claim 10, further comprising a memory, wherein the processor is configured to store sample data associated with the detection signal in the memory.

14. The apparatus of claim 11, wherein the first biological analyte includes ω-3 fatty acids and associated reference data is stored in the memory.

15. The apparatus of claim 14, wherein the second biological analyte is selected from a group consisting of ω-6 fatty acids and ω-9 fatty acids and associated reference data is stored in the memory.

16. The apparatus of claim 11, wherein at least one of the first and second biological analytes is selected from the group consisting of lipids, glycosylated proteins, cholesterol, blood glucose, and triglycerides, and associated reference data is stored in the memory.

17. The apparatus of claim 10, further comprising a communication interface configured to receive dietary supplement data associated with the first biological analyte, wherein the display is configured to display a supplement recommendation based on the dietary supplement data and the health indication associated with the concentration of the first biological analyte.

18. The apparatus of claim 11, wherein the processor is configured to determine a ratio of concentrations of the first and second analytes.

19. The apparatus of claim 10, wherein the radiation source is configured to emit near infrared radiation, and the in vivo sample is situated to reflect or transmit the detected radiation to the detector.

20. The apparatus of claim 10, further comprising a sample holder configured to the position the in vivo sample with respect to the radiation source and the detector.

21. The apparatus of claim 10, wherein the processor is configured to determine the concentrations based on emitted radiation in a wavelength range between about 1150 nm and 1190 nm.

22. The apparatus of claim 11, wherein the processor is configured to determine a ratio of the concentrations of the first analyte and the second analyte based on emitted radiation in a near infrared wavelength range.

23. A method, comprising:
non-invasively scanning a living organism to determine an amount of two or more biological analytes with respect to reference amounts of the two or more analytes;
obtaining an indication of an amount of at least one of the biological analytes that is available in an ingestible substance; and
providing a dietary recommendation for the living organism relating to ingestment of the ingestible substance by the living organism based on the amount of the two or more biological analytes and the obtained indication.

24. The method of claim 23, wherein the indication of the amount of at least one of the biological analytes that is available in the ingestible substance is obtained by communication via a wide area network.

25. The method of claim 23, wherein the indication of the amount of at least one of the biological analytes that is available in an ingestible substance is retrieved from a computer readable medium.

26. The method of claim 20, wherein the dietary recommendation is associated with at least one of ω-3, ω-6, and ω-9 fatty acids.

27. The method of claim 20, wherein the dietary recommendation is associated with at least one analyte selected from the group consisting of lipids, glycosylated proteins, cholesterol, blood glucose, and triglycerides.

28. The method of claim 21, further comprising scanning the living organism after ingestment of the ingestible substance so as to determine effectiveness of the dietary recommendation.

29. A method, comprising:
providing an analyte measurement apparatus to a dietary supplement customer;
producing a supplement recommendation based on in vivo assessment of a plurality of analytes produced by the apparatus; and
providing supplements based on the supplement recommendation.

30. The method of claim 29, wherein at least one of the plurality of analytes is selected from the group consisting of lipids, glycosylated proteins, and cholesterol.

31. The method of claim 29, wherein the plurality of analytes include lipids, glycosylated proteins, and cholesterol.

32. The method of claim 29, further comprising providing a health indicator based on the in vivo assessment.

33. The method of claim 29, further comprising transmitting the in vivo assessment of the plurality of analytes to a supplement supplier, wherein the supplement supplier provides the supplements based on the supplement recommendation.

34. The method of claim 29, further comprising producing the in vivo assessment based on at least one near infrared spectrum.
